(19) United States
(12) Patent Application Publication
(10) Pub. No.: US 2020/0316255 A1
(43) Pub. Date: Oct. 8, 2020

(54) HUMAN CELL-DEPOSITED EXTRACELLULAR MATRIX COATINGS FOR TEXTILES AND FIBERS

(71) Applicant: THE SECANT GROUP, LLC, Telford, PA (US)

(72) Inventors: Brian GINN, Chalfont, PA (US); Peter D. GABRIELE, Frisco, TX (US); Jeremy J. HARRIS, Doylestown, PA (US)

(21) Appl. No.: 16/839,347

(22) Filed: Apr. 3, 2020

Related U.S. Application Data
(60) Provisional application No. 62/828,604, filed on Apr. 3, 2019.

(51) Int. Cl. A61L 27/36; C12M 3/00; A61L 27/58; A61L 27/24; C12M 1/12

(52) U.S. Cl. CPC A61L 27/3633; C12M 21/08; A61L 2420/04; A61L 27/24; C12M 25/14; A61L 27/58

(57) ABSTRACT

A process of forming a coated textile includes culturing human cells on a fiber of a textile such that the human cells produce and deposit human extracellular matrix (hECM) on the textile. The process also includes removing the human cells from the hECM to provide the coated textile of the textile and a coating comprising a residual of the hECM produced and deposited by the human cells on the textile during the culturing. A coated textile includes a textile and a coating on the textile. The coating includes hECM in a cell-deposited state in the coating. A solid-state bioreactor composition includes a poly(glycerol sebacate) (PGS) adduct. The PGS adduct includes PGS and a promoting factor or a promoting factor precursor. Another method includes implanting a coated textile in a human. The coated textile is an autograft. The coating includes hECM deposited by human cells from the human.

### CROSS-REFERENCE TO RELATED APPLICATIONS

This application claims priority to and the benefit of U.S. Provisional Application No. 62/828,604 filed Apr. 3, 2019, which is hereby incorporated by reference in its entirety.

### FIELD OF THE INVENTION

The present disclosure is generally directed to bio-compatibilization of biomedical materials. More specifically, the present disclosure is directed to the usage of primary human cells or cell lines to generate human extracellular matrix (hECM)-coated woven, braided, non-woven, or knit biomedical textiles or fibers for use as or in an implantable medical device, component, or scaffold in vivo, a bioreactor scaffold for in vitro cell expansion or stem cell differentiation, or a substrate for routine cell culture applications.

### BACKGROUND OF THE INVENTION

Many implantable devices generate an inflammatory response due to a lack of human-specific signaling moieties that help the body recognize the implant as human-compatible. The increased incidence of elevated immune system response reduces performance and lifetime of the implant while simultaneously slowing healing of the patient due to extended inflammation of the local microenvironment at the site of implantation.

Extracellular matrix (ECM), produced by certain cells, provides structural and biochemical support in vivo to surrounding cells. ECM used in biomedical devices conventionally comes from decellularized tissue, where the ECM components are then pulverized and used as a powder or formed into an isotropic hydrogel material for application to a biomedical product. While this process presents the ECM components that provide native adhesive cues and cytokines, it does not match the native underlying ECM structure and hence provides a less-than-ideal protection against an inflammatory response.

To improve cellular binding, xenogenic ECM components, such as, for example, bovine-derived collagen, are frequently used as structural or coating materials in products used for cell culture or implantation. Xenogenic materials, however, also elicit an immune response to the implanted device. When such components are used as tissue culture substrates in vitro, through products, such as, for example, a gelatinous protein mixture secreted by Engelbreth-Holm-Swarm (EHS) mouse sarcoma cells and marketed under the trade name Matrigel by Corning Life Sciences (Tewksbury, Mass.) and BD Biosciences (San Jose, Calif.), there is often a disconnect between the benchtop in vitro studies and the clinical application due to species mismatch at both ends of the product development cycle. This can lead to poor clinical trial results and failed product development cycles.

In order to match the native tissue architecture and innate signaling cues, some medical devices are generated through the decellularization of ECM, which can be structurally weak and fail during handling by the surgeon. These decellularized devices typically rely on the use of donor tissue or cadaveric tissue, both of which are only available in a very limited supply.

U.S. Pat. No. 7,795,027, entitled “Extracellular matrix composite materials, and manufacture and use thereof”, issued Sep. 14, 2010 to Hiles, discloses using human cells of various types to secrete human ECM on implantable devices. Hiles utilizes a process where the implantable device is first coated in a xenogenic ECM, upon which human cells are then cultured and later removed after secreting a top layer of human ECM. The underlying xenogenic material, however, may still elicit an inflammatory response.

What is needed is an implantable device that is coated to reduce or eliminate an inflammatory response from the human host.

### BRIEF DESCRIPTION OF THE INVENTION

Exemplary embodiments are directed to textiles coated with human extracellular matrix material in a cell-deposited state and processes of forming and using such coated textiles.

According to an exemplary embodiment, a process of forming a coated textile includes culturing human cells on at least one fiber of a textile such that the human cells produce and deposit human extracellular matrix (hECM) on the textile. The process also includes removing the human cells from the hECM to provide the coated textile of the textile and a coating including a residual of the hECM produced and deposited by the human cells on the textile during the culturing.

According to another exemplary embodiment, a coated textile includes a textile and a coating on the textile. The coating includes hECM. The hECM is in a cell-deposited state in the coating.

According to yet another exemplary embodiment, a solid-state bioreactor composition includes a poly(glycerol sebacate) adduct. The poly(glycerol sebacate) adduct includes poly(glycerol sebacate) and a promoting factor or a promoting factor precursor.

According to another exemplary embodiment, a method includes implanting a coated textile in a human. The coated textile is an autograft including a textile and a coating on the textile. The coating includes human extracellular matrix (hECM) in a cell-deposited state deposited by human cells from the human.

Various features and advantages of the present invention will be apparent from the following more detailed description, taken in conjunction with the accompanying drawings which illustrate, by way of example, the principles of the invention.

### BRIEF DESCRIPTION OF THE DRAWINGS

FIG. 1 schematically shows the L-carnitine shuttle of the Krebs cycle.

FIG. 2 shows an uncoated textile.

FIG. 3 shows the textile of FIG. 2 with a coating including human extracellular matrix material in an embodiment of the present disclosure.

FIG. 4 shows FTIR spectra of the uncoated textile of FIG. 2, the coated textile of FIG. 3, and a textile with lyophilized cells.

FIG. 5 shows the 900 to 1100 cm⁻¹ wavenumber region of the spectra of FIG. 4.

FIG. 6 shows the 1500 to 1800 cm⁻¹ wavenumber region of the spectra of FIG. 4.

FIG. 7 shows the 2700 to 3000 cm⁻¹ wavenumber region of the spectra of FIG. 4.

FIG. 8 shows cell counts during various stages of cell culture on fibers.

FIG. 9 shows cell count ratios for the cell cultures of FIG. 8.

FIG. 10 shows spreading of cardiac fibroblasts on an ECM-coated textile.

Wherever possible, the same reference numbers will be used throughout the drawings to represent the same parts.

### DETAILED DESCRIPTION OF THE INVENTION

Provided herein are compositions and processes of forming compositions including coatings of human extracellular matrix (hECM) components in a cell-deposited state on a substrate.

In exemplary embodiments, the hECM components are in a cell-deposited state based on deposition through cell culture of human ECM-producing cells. The substrate for the coating may be any appropriate textile or fiber material. The coating of hECM through cell culture may be performed on either a stock yarn or fiber or the finished textile.

Appropriate textiles may include any textile material of construction that may be suitably used for implantable non-resorbable textiles, including, but not limited to, poly(ethylene terephthalate) (PET), polytetrafluoroethylene (PTFE), polyolefins, such as, for example, polypropylene, polyethylene, or polyethylene vinyl acetate, or collagen, as well as a variety of resorbable materials, including, but not limited to, poly(glycolic acid) (PGA), poly(glycerol sebacate) (PGS), poly(lactic acid) (PLA), poly(lactic-co-glycolic acid) (PLGA), poly(trimethylene carbonate) (PTMC) or polycaprolactone (PCL) that may be interwoven, for example, with more permanent materials such as PET and PTFE.

The human cells may be any appropriate human cells that produce and deposit extracellular matrix. In some embodiments, the human cells are human fibroblast cells, smooth muscle cells, osteocytes, cardiomyocytes, or chondrocytes. In a preferred embodiment, the human cells are from the same individual in whom the textile is to be implanted, resulting in self-recognized ECM and giving the textile an autograft-like characteristic. In other embodiments, the cells are not patient-specific. In such embodiments, the human cells may be from a different human than the human in whom the textile is to be implanted, giving the textile an allograft-like characteristic. In preferred embodiments, the coating is free of xenogenic material.

The hECM material may be any appropriate hECM material, including, but not limited to, proteoglycans, such as, for example, heparan sulfate, chondroitin sulfate, or keratan sulfate; non-proteoglycan polysaccharides, such as, for example, hyaluronic acid; proteins, such as, for example, collagen or elastin; cell-adhesion proteins, such as, for example, fibronectin or laminin; or combinations thereof.

As used herein, “cell-deposited state” refers to a structure and morphology of extracellular material produced by deposition from cells.

As used herein, “doped” or “doping” refer to a promoting factor added to a treatment or in a process.

As used herein, “encapsulation” refers to sphericalization of a delivery structure by a PGS microsphere process. In some embodiments, the PGS microspheres are formed by a process as disclosed in U.S. Patent Application Publication No. 2018/0280912, entitled “Cured Biodegradable Microparticles and Scaffolds and Methods of Making and Using the Same”, by Lu et al., published Oct. 4, 2018, which is incorporated by reference herein in its entirety.

As used herein, “treated surface” refers to any surface treated with an ECM coating and referenced or identified on an implantable article.

As used herein, “extrudate” refers to any material or article of manufacture that is extruded or shaped by force through a die or other suitable orifice with or without head.

As used herein, “xenogenic” refers to any material not formed by cells of the target species for the implant, such as non-human for a human patient.

As used herein, “allograft” refers to any material formed by cells of the target species but not the target individual for the implant.

As used herein, “autograft” refers to any material formed by cells of the target individual for the implant.

In some embodiments, human signaling factors are provided through the culturing of human cells on the surface of biomedical textiles and fibers that deposit species-matching extracellular matrix (ECM), which promotes recognition of the implant as “human” to reduce the inflammatory response and speed healing. In some embodiments, these human signaling factors are captured by the secreted extracellular matrix. In some embodiments, the deposited ECM may include proteinaceous components, which may include, but are not limited to, collagen or fibrin. In some embodiments, the deposited ECM may include glycosaminoglycans, which may include, but are not limited to, hyaluronic acid, chondroitin sulfate, keratin sulfate, or heparin. In some embodiments, the deposited ECM may contain growth factors, which may include, but are not limited to, vascular endothelial growth factor (VEGF), epidermal growth factor (EGF), insulin-like growth factor (IGF), interleukins (for example, but not limited to IL-2, IL-4 and IL-5), or platelet-derived growth factor (PDGF).

In some embodiments, the addition of human ECM components to the textile surface reduces or eliminates potential issues of species mismatch observed with xenogenic materials.

With the deposition of ECM on textiles and fibers by human cells, a good analog of the ECM surface moieties present in the native human tissue is generated that provides improved structural integrity to the deposited ECM without the need for donor tissue from the patient or a cadaveric source. In some embodiments, cells are harvested from the patient to make an autograft-like version of an ECM-coated textile or fiber. Similarly, primary human cells or cell lines from other sources may be used for generating an off-the-shelf, ECM-coated product for applications in which allograft-like scaffolds are effective.

In some embodiments, human ECM-secreting cells are cultured directly on an unmodified textile or fiber such that there is no xenogenic precoating or any other xenogenic components and/or the coated textile or fiber is free of xenogenic material. In exemplary embodiments, the only biological material present in or on the coated textile or fiber is a product of human cells or the human ECM-secreting cells. In other words, the coated textile or fiber is free of biological material not derived from human cells or the human ECM-secreting cells.

In many parts of the world, there are cultural and/or religious barriers preventing the use of an implantable device that includes xenogenic material or even allograft material. In addition to the innate non-immunogenic advantages of including only autograft materials, exemplary embodiments also overcome these cultural and/or religious barriers. In some embodiments, the autograph implant may be considered to provide a self-therapy.

In some embodiments, the underlying textile or fiber is pre-conditioned prior to human cell seeding through surface modification, such as plasma treatment, acid/base treatment, or mechanical deformation to promote improved cellular attachment and ECM-secretion.

In some embodiments, a non-xenogenic resorbable coating, such as PGS, is applied to the surface of the textile or fiber prior to seeding with cells to improve cell ECM-secretion. The non-xenogenic resorbable coating serves as a primer coat to better compatibilize the surface for the cell culture and for receiving the coating of hECM. When the non-xenogenic resorbable coating includes PGS, the PGS may provide nutrients to the cells of the cell culture and may also upregulate one or more genes associated with production and secretion of one or more hECM molecules by the human ECM-secreting cells.

In some embodiments, the bio-interfacing of textile and fiber surfaces used as components in implantable devices and cell culture technologies is enhanced by presenting the innate biochemical and structural cues associated with human ECM that help the body recognize “self”, thereby improving the biological performance of biomedical textiles and fibers and reducing the inflammatory response to these materials.

In some embodiments, a process includes extracting fibroblastic or other hECM-producing cells from a patient and culturing those cells on a textile or fiber structure in vitro. The extraction preferably includes separating and purifying the hECM-producing cells from other cells and any extracellular or other tissue material such that it is not cell tissue that is applied to the textile or fiber. Instead, the hECM-producing cells may be the only cellular material exposed to the textile or fiber. The subsequent culturing is preferably a cell culturing and not a tissue culturing on the textile or fiber. In some embodiments, the cell culture is contained within a wave-mixed bioreactor bag system. Once sufficient time has passed for fibroblast ECM deposition to coat and/or cover the textile or fiber surface, the process includes removing the fibroblasts to yield a decellularized textile or fiber surface having a coating of patient-specific ECM to improve cellular integration upon implantation. In some embodiments, the decellularized coated textile or fiber is free of cells.

In some embodiments, a process utilizes stem cell-derived cells from a patient for ECM deposition on a textile or fiber.

In some embodiments, a process utilizes a mixed population of human cells for ECM deposition on a textile or fiber.

In some embodiments, a process utilizes fibroblasts specific to the target implant site, such as, for example, dermal fibroblasts for skin care applications or cardiac fibroblasts for cardiovascular grafts and patches.

In some embodiments, a process utilizes human cells that are non-patient specific for generation of off-the-shelf hECM enhanced textile or fiber products after ECM deposition by the human cells and subsequent cell removal.

In some embodiments, a process includes leaving a patient's own cells on a previously implanted and subsequently removed textile or fiber for reimplantation as a tissue-engineered product. In some embodiments, the textile or fiber is reimplanted after genetic modification or differentiation. In some embodiments, a process includes further processing an hECM-enhanced surface of a textile or fiber with cell-deposited ECM through a secondary culture of endothelial cells to pre-vascularize the textile or fiber prior to implantation or reimplantation.

In some embodiments, a process incorporates other cell signaling factors, such as, for example, growth factors or cell capture agents, such as, for example, heparin, onto an hECM-enhanced textile or fiber surface prior to implantation, after removal of cells.

In some embodiments, a process includes treating the cells cultured with the textiles or fibers with one or more ECM production-accelerating factors, such as, for example, ascorbic acid (Vitamin C), in a culture media for fibroblasts.

In some embodiments, the process does not include or require forming a preliminary xenogenic or allograft ECM coating and the end product use is flexible in its application toward individual textile or fiber components, implantable textiles or fibers as a device, or in an in vitro culture scaffold. Additionally, the coated textile or fiber may be used in an autograft-like or allograft-like mode depending on the cell source. The high surface area presented by the coated textile or fiber surface better promotes “human-like” recognition of the textile or fiber by the body.

In some embodiments, a process matches the biological structural orientation through the hECM deposited by human cells on the textile or fiber surface that provides a composite structure easily addable to other medical devices as a secondary component to enhance tissue integration or as an easy-to-surgically-handle implantable device, such as, for example, a mesh or patch.

In some embodiments, a process provides the capacity to not only match the correct species by using human cells but also to match the cells found in the tissue of interest to generate a personalized scaffold tailored for specificity towards the implantation site to enhance healing. Typical implantable bio-textile or bio-fiber applications rely on cellular infiltration and in situ ECM deposition by the host cells to drive tissue integration, whereas compositions and processes of the present disclosure accelerate the integration process by providing a pre-prepared hECM template to the host tissue to provide one or more host recognition signaling moieties that are lacking in current implantable textile or fiber products.

In some embodiments, a coated textile or fiber is part of an implantable textile or fiber application and those involving fiber production used for textile generation or other fibrous applications, such as, for example, sutures. Braided, woven, knit, and non-woven textile scaffolds may be prepared using conventional textile preparation processes, with mechanical and biodegradation properties being engineered as appropriate for their downstream applications. In some embodiments, human cells, such as, for example, fibroblasts, are taken from a patient through biopsy, or non-patient-derived cells may be obtained from a secondary commercial source. For those taken from the patient, the hECM-producing cells may be isolated from other tissue substituents or other cells by a cell sorting technology, such as, for example, flow cytometry or antibody-based processes. The hECM-producing cells may then be seeded onto sterilized textile scaffolds and cultured for a predetermined culture time, such as, for example, in the range of 2 to 28 days, alternatively 2 to 3 days, alternatively 2 to 7 days, alternatively 7 to 14 days, alternatively 14 to 28 days, or any range or sub-range therebetween, to proliferate and produce hECM associated with the target implant site tissue. The culture time may depend on the amount of biopsied material and the size of the textile scaffold for reimplantation. The modified textiles or fibers may then be freeze-dried to preserve the cytokines captured in the hECM layer and then either implanted directly into a patient or incorporated into a medical device as a secondary component to enhance tissue integration.

In some embodiments, upon cell culture completion, cells may be removed from the hECM-modified textile or fiber surface by enzymatic treatments, such as, for example, through the use of trypsin or dispase. In other embodiments, specific enzymatic treatments are used to digest specific ECM components during removal of cells, such as collagenase to remove collagen or elastase to remove elastin. In yet other embodiments, chemical treatments are used to remove the cells from the deposited ECM, such as through use of detergents, acidic treatments, or base treatments. In some embodiments, chelating agents, such as, for example, ethylenediaminetetraacetic acid (EDTA), are added to the cell removal solution to remove the metallic ions that are used by cells for cell-substrate or cell-cell binding. In other embodiments, cyclical freezing and thawing is used to kill cells for removal from the ECM. In other embodiments, cells are subjected to electrical forces to electroporate the cell membrane to kill cells for removal from the secreted ECM. In an additional embodiment, apoptosis of cells is induced to promote their removal from the secreted ECM through addition of one or more soluble apoptosis-inducing factors such as, for example, paclitaxel, camptothecin, etoposide, or doxorubicin hydrochloride. In some embodiments, an endonuclease, such as, for example, benzoase, is added to the solution to break down and remove residual deoxyribonucleic acid (DNA) and ribonucleic acid (RNA) from the cell-ECM secreted coated textile or fiber.

In some embodiments, the human fibroblasts are obtained through commercial sources and cultured to generate an hECM coating on a textile or fiber in an allograft-like off-the-shelf product. Such a configuration is expected to have a substantially better bio-interfacing performance compared to untreated textiles or fibers or those coated with a xenogenic ECM agent, as the acellular hECM textile lacks the foreign ECM that might induce a negative biological response.

Further advancements in personalized medicine workflows through improved biocontainment systems designed specifically for textile use, such as those made via single-use bioreactor cell expansion bags, enable the application of a patient's own cells towards the generation of the hECM coatings on the textile or fiber. Dermal fibroblasts may be preferred and sufficient for most autograft applications, as they have the greatest ease of acquisition from a patient.

While it is possible to harvest cells from a specific patient tissue, there may be difficulty in obtaining tissue-matching cells from locations like those for cardiovascular applications, where it would be preferable to have any harvest procedure be as minimally invasive as possible. There may be opportunities in the future to use more easily-acquired stem cell-derived sources to generate specific tissue ECM-producing cells, such as using adipose-derived stem cells (ADSCs) to produce specific “strains” of ECM-producing cells like fibroblasts for tissues like cardiac, where a surgeon might not wish to remove any cardiac tissue to obtain fibroblasts.

The textiles or fibers to be coated may be woven, braided, knit, and/or non-woven. The fibers of the textile to be coated may be varied in denier and filament type, such as, for example, single filament, multifilament, texturized fibers, yarn twist, or ply. The fibers of the textile to be coated may have any of a number of appropriate cross-sectional geometries, such as, for example, circular, cross-shaped, flat, oval, multi-lobal, rectangular, or square.

A textile scaffold to be coated may be generated using fibers composed of alternative synthetic materials, including, but not limited to, PET, PCL, poly(lactic-co-glycolic acid) (PLGA), polypropylene (PP), or poly(vinylidene fluoride) (PVDF). Alternatively, a textile scaffold to be coated may be generated using fibers composed of alternative biologically-derived materials, such as, for example, collagen, fibrin, alginate, chitosan, or silk. Alternatively, a textile scaffold to be coated may be generated using blends of synthetic-synthetic polymers, synthetic-biological polymers, or biological-biological polymers.

A textile scaffold to be coated may be additionally seeded with tissue-specific support cells to assist fibroblasts with ECM production, such as, for example, chondrocytes for cartilage wrap products or keratinocytes for dermal patches, to further tune the ECM surface and deposit cytokines specific to the target tissue or implantation location of the coated product.

The cell culturing on the textile scaffold may be done, for example, in a flask, in a dish, on a multi-well plate, in a soft plastic biocontainment bag, or in a bioreactor system. The culture time may be adjusted to tune the density/thickness of the hECM layer.

In some embodiments, additional cell adhesion ligands are incorporated into the hECM layer to further tune the tissue and cell integration.

In some embodiments, multiple sequential layers of differing ECM are deposited by first culturing one cell type followed by culturing a second, different type of cell after removal of the first to recapitulate a naturally-occurring biological structural order, such as, for example, a dense basal lamina layer (first layer) supporting a functional ECM layer (second layer).

In some embodiments, an ECM coating produced by fibroblasts directly on a textile or fiber improves the biocompatibility of the textile or fiber relative to the textile or fiber without the ECM coating, with an ECM coating not produced by fibroblasts directly on the textile or fiber, or with a non-human ECM coating.

In some embodiments, a process co-cultures a cell line with an article. The engineered form of textile or fiber may be assembled as either resorbable or non-resorbable. The surface of the textile or fiber may be pre-treated as needed to allow for a “tie” or “kiss” layer of a biodegradable polymer, such as, for example, a glycerol-sebacate-containing polymer, such as, for example, PGS, lysine-poly(glycerol sebacate) (KPGS), poly(glycerol sebacate urethane) (PGSU), or another PGS-adduct. The PGS-containing layer may be formulated as a nutrient support option. An adduct of PGS and a cell adhesion promoter may be included to enhance cell adhesion to the surface. Appropriate cell adhesion promoters may include, but are not limited to, arginine-glycine-aspartic acid (RGD), isoleucine-lysine-valine-alanine-valine (IKVAV), or tyrosine-isoleucine-glycine-serine-arginine (YIGSR), peptides associated with cell adhesion to ECM. In some embodiments, the process includes exposing the article to a tissue-specific fibroblast line, culturing and expanding the tissue-specific fibroblast line to produce and deposit ECM material, and harvesting the tissue-specific fibroblast line to decellularize the article. The process may further include securing and/or fixing a fragile or weak ECM coating to the article with a fog of PGS or other suitable bio-adherent or mortar.

In some embodiments, the PGS-containing layer goes beyond providing nutrient support by providing one or more promoting factors or promoting factor precursors, such as, for example, cofactor or coenzyme moieties, in forming a solid-state bioactive surface. In some embodiments, the PGS-containing layer includes at least one promoting factor or promoting factor precursor, such as, for example, one that aids in driving the Krebs cycle, also known as the citric acid cycle (CAC) or the tricarboxylic acid (TCA) cycle, in the mitochondria of the cells cultured on a surface containing the PGS-containing layer. In some embodiments, the cultured cells are autograft cells. The Krebs cycle is important to cell metabolism and consequently to the health and pathology of disease. The Krebs cycle results in the oxidation of nutrients to produce usable chemical energy for the cells.

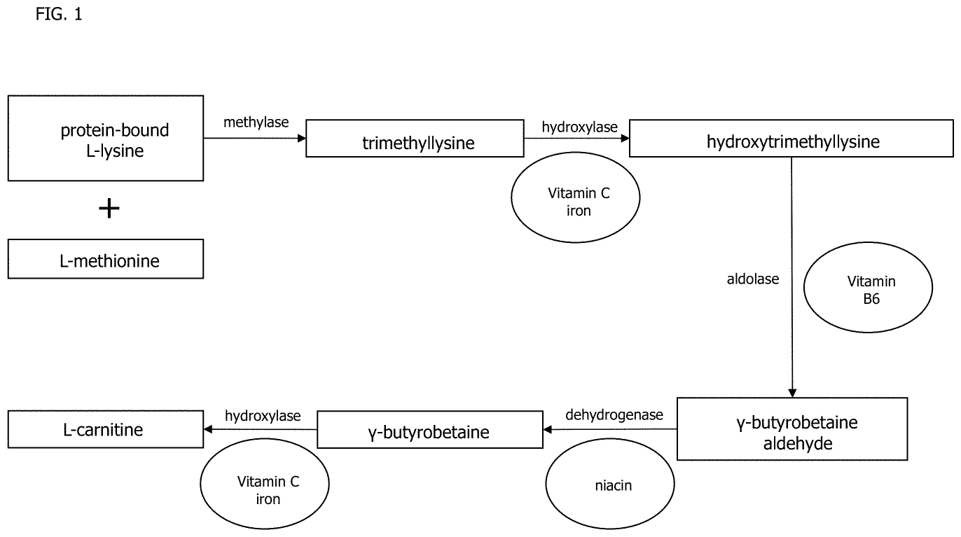

The dominate source of energy production in the Krebs cycle comes from fatty acids, not sugars. The fatty acid is a carbon source, and consequently, the fatty acid pathway is important to cellular bioenergetics. Important promoting factors to the fatty acid metabolic path are Coenzyme Q₁₀ (CoQ₁₀), the amino acid L-carnitine, and calcium-magnesium-ATPase. L-carnitine is part of the fatty acid shuttle system that works in concert with CoQ₁₀ in the Krebs cycle, as shown in FIG. 1.

As shown in FIG. 1, trimethyllysine is formed from protein-bound L-lysine and L-methionine in the presence of a methylase. Hydroxytrimethyllysine is formed from trimethyllysine in the presence of Vitamin C, iron, and a hydroxylase. γ-butyrobetaine aldehyde is formed from hydroxytrimethyllysine in the presence of Vitamin B₆ and an aldolase. γ-butyrobetaine is formed from γ-butyrobetaine aldehyde in the presence of niacin and a dehydrogenase. L-carnitine is formed from γ-butyrobetaine in the presence of Vitamin C, iron, and a hydroxylase.

CoQ₁₀, also known as ubiquinone, ubiquitous in eukaryotic cells, and essential to mitochondrial health, has the following chemical structure:

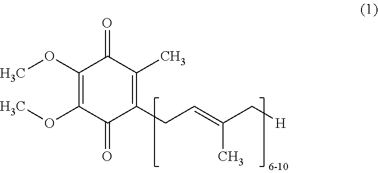

L-carnitine, a quaternary amino acid synthesized in the body from the promoting factor precursor lysine, has the following chemical structure:

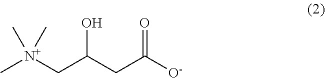

In some embodiments, a coating or scaffold material includes one or more carbon sources, such as, for example, glycerin and/or sebacic acid in PGS or a PGS adduct, as nutrient supports that feed the Krebs cycle and one or more promoting factors or promoting factor precursors, such as, for example, CoQ₁₀, L-carnitine, lysine, calcium, and/or magnesium, that drive the Krebs cycle, thereby promoting energy generation by the cultured cells and improving cultured cell health and ECM production.

In some embodiments, different PGS-adducts, including specific cofactor/coenzyme moieties as promoting factors or promoting factor precursors, are co-blended into a single vehicle film.

In some embodiments, different PGS-adducts are layered, and polymer flow produces comingling of the layers.

In some embodiments, a coating or scaffold material includes a solid-state bioreactor composition including the combination of a KPGS adduct, a PGS-magnesium (PGS-Mg) adduct, and CoQ₁₀. Since it does not have a reactive group for PGS addition, CoQ₁₀ may be compounded into the solid-state bioreactor composition. Such a solid-state bioreactor composition may serve in any of multiple applications, including, but not limited to, addition to textile substrates for either bioreactor design or implant coatings.

In some embodiments, the rate of fatty acid synthesis by cells following PGS breakdown is increased, because the cell energy to generate the promoting factor is reduced or eliminated by the presence of the promoting factor or promoting factor precursor. In some embodiments, a series of PGS-adducts are made separately and co-blended based on cell requirements for hECM production in order to increase hECM production, thereby customizing cell energetic requirements. In some embodiments, the cultured cells are autograft cells such that the resulting hECM has an autograft character. In some embodiments, cardiomyocytes and/or liver cells, having higher energy demands than skin cells, may need higher levels of one energy-driving adduct over another to keep the cells at optimal burn rates.

In some embodiments, a process includes preparing an extrudate surface prior to cell culturing. The extrudate may be a fiber, film-molded shape, die-cut, template, or any configuration so desired to engineer the implantable device. The extrudate surface may be post-processed to pre-activate or treat the surface for tie, kiss, or direct cell deposition. In some embodiments, the preactivated deposition may include one of the cell adhesion promotors RGD, IKVAV, or YIGSR, or a PGS adduct of RGD, IKVAV, or YIGSR. The extrudate may be seeded with one or more appropriate cell lines.

In some embodiments, a process includes microsphere encapsulation or surface attachment of a cell line. The cells may be pre-encapsulated within PGS microsphere technology to be directly delivered to a treated surface. The pre-encapsulated cells may be directly delivered to a surface via a suitable vehicle and/or via a suitable deposition technique. The pre-encapsulated cells may be formulated into a treatment coating.

In some embodiments, a process includes activating resorbable and non-resorbable fibers in a wet state, under melt-flow, or under electrospinning. A fiber stock may be spin-finished with activating treatment by any of a plurality of deposition processes, including, but not limited to, spraying, dipping, and/or coating. The fiber resin stock may be modified or co-compounded with a nutrient supplement. The fiber article of manufacture may employ islands-in-the-sea to form a hybrid doped composition, which may include non-resorbable islands in a resorbable sea, resorbable islands of differential degradation rates in a resorbable sea, or non-resorbable islands-in-the-sea with a sea surface modified to accept cell contact, adhesion, and/or colonization. The textile, fiber, or engineered article may be fabricated or formed from a modified raw material to enhance cell-surface interactions. The textile fiber may be formed with a modifying treatment, such as, for example, a nutrient-doped spin-finish, direct affixation or deposition of cells onto a fiber surface during spin finishing, or aerosol co-deposition of cells during electrospinning.

In some embodiments, a process includes depositing cells or collagen on a surface of an article. Appropriate low-shear and low-heat deposition processes include, but are not limited to, spraying, coating, spinning, fogging, aerosolizing, and gravity. A textile fabric may be designed as a filtration structure, whereby suspended cultures with viable cell lines are entrapped onto and within fiber structures of the textile fabric during a filtration action. The textile structure may be pre-treated or doped with a surface-activating treatment and the article may be dip-coated. A finished fiber stock, textile, or extrudate article of manufacture including a collagen surface coordinating moiety may be co-cultured (co-incubated) with a recombinant organism capable of producing recombinant collagen such that the produced polypeptide binds or precipitates onto the surface of the co-cultured (co-incubated) article without microbe deposition or incorporation.

In some embodiments, an ECM-coated textile or fiber is evaluated by characterizing the ECM deposition and/or a response to the deposited ECM coating the textile or fiber.

In some embodiments, a process includes culturing human fibroblasts on a woven, braided, or knit textile to form ECM on the surface of the textile. Fibroblasts are cultured for one week on textiles to generate and deposit human ECM on the textile surface. The fibroblasts are then removed through trypsinization. The coated textiles may be washed with surfactant to remove soluble cytokine proteins bound in the deposited ECM. Alternatively, if the presence of soluble cytokine proteins is beneficial to the downstream application, the coated textile may be left unwashed. The coated textile may be freeze-dried or used while still wet for further processing or evaluation. The ECM deposition may be evaluated through histological staining, electron microscopy, or infrared spectroscopy of treated textiles in comparison to untreated textiles.

In some embodiments, a secondary cell type, such as human mesenchymal stem cells, is subsequently seeded on a textile surface with deposited hECM and cultured for one week followed by testing to evaluate whether the pre-deposition of species-matching hECM improves cell response to the textile. The testing may include comparing a cell response to an hECM-coated textile to a cell response to an uncoated textile. In some embodiments, the proliferation rates are evaluated by a 3-(4,5-dimethylthiazol-2-yl)-2,5-diphenyltetrazolium bromide (MTT) assay, a 2-(4-iodophenyl)-3-(4-nitrophenyl)-5-(2,4-disulfophenyl)-2H-tetrazolium, monosodium salt (WST) assay, or an alamarBlue® assay (AccuMed International, Inc., Chicago, Ill.). Other evaluation techniques may include, but are not limited to, a cell count to assess attachment efficacy, a live/dead assay to assess cell viability, fixing and staining cells to look at cellular morphology differences at early and late culture timepoints, and an enzyme-linked immunosorbent assay (ELISA) specific to a cytokine of interest to assess production of cytokines by cells. An improvement in the speed of proliferation and in attachment is expected when pre-deposited hECM is present.

In comparative embodiments, a secondary cell type, such as human mesenchymal stem cells, is subsequently seeded on a textile surface with deposited non-human ECM, such as, for example, ECM deposited by rat fibroblasts, and cultured for one week followed by testing to evaluate whether the pre-deposition of species-mismatching ECM affects the cell response to the textile. The testing may include comparing the cell response to a non-human ECM-coated textile to the cell response to an hECM-coated textile. In some embodiments, the proliferation rates are evaluated by an MTT assay, a WST assay, or an alamarBlue® assay. Other evaluation techniques may include, but are not limited to, a cell count to assess attachment efficacy, a live/dead assay to assess cell viability, fixing and staining cells to look at cellular morphology differences at early and late culture timepoints, and ELISA specific to a cytokine of interest to assess production of cytokines by cells. A reduced performance is expected when pre-deposited non-human ECM is present.

In addition to the previously-described applications, a cell-deposited ECM coating may have applications other than as part of a human implant.

In some embodiments, the coated textile or fiber is formed for veterinary applications using non-human species-specific cells, which may include, but are not limited to, canine cells, feline cells, porcine cells, equine cells, or avian cells.

In some embodiments, the coated textile or fiber serves as a tear away top and bottom starter layer for existing lab-grown meat products, such as, for example, a ground beef or hamburger substitute, like the paper sheets used to divide deli cheese slices at a grocery store.

In some embodiments, the coated textile or fiber is applied towards generating anisotropic tissue scaffolds, such as, for example, when the textile or fiber has a preferred orientation for applications, such as, for example, peripheral nerve grafts or cardiac patches.

In some embodiments, the coated textile or fiber is used as a wound sealant or filler material in the treatment of serious burn wounds to promote cellular re-infiltration.

In some embodiments, the coated textile or fiber is applied in the dermocosmetic industry for in vitro screening of various factors and formulations on human collagen found in skin, such as, for example, anti-aging creams or effects of ultraviolet (UV) light on skin, with an hECM-coated textile serving as a desirable alternative to testing on animals.

### EXAMPLES

The invention is further described in the context of the following examples which are presented by way of illustration, not of limitation.

### Example 1

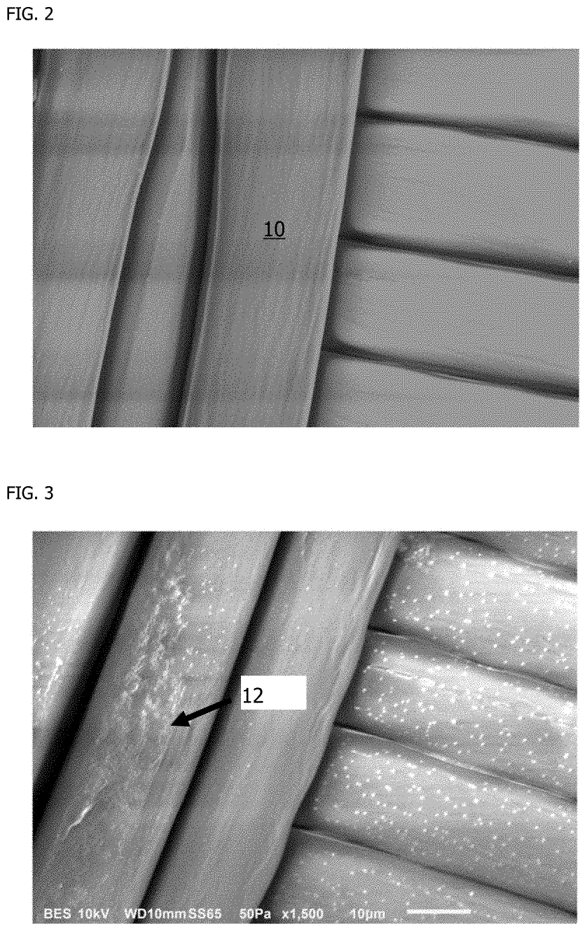

Human cardiac fibroblast cells were cultured on a woven PVDF textile for six days of cell culture followed by induction of cellular apoptosis using UV light and saline washing to remove cells. FIG. 2 shows the woven PVDF textile 10 after manufacture and prior to cell culture. The woven PVDF textile 10 does not have a coating of ECM present prior to seeding cells for culture. FIG. 3 shows the woven PVDF textile after human cardiac fibroblast cell culture and cell removal. The visible texture on the woven PVDF textile in FIG. 2 indicates the presence of human cardiac fibroblast cell-deposited ECM 12 on the textile.

### Example 2

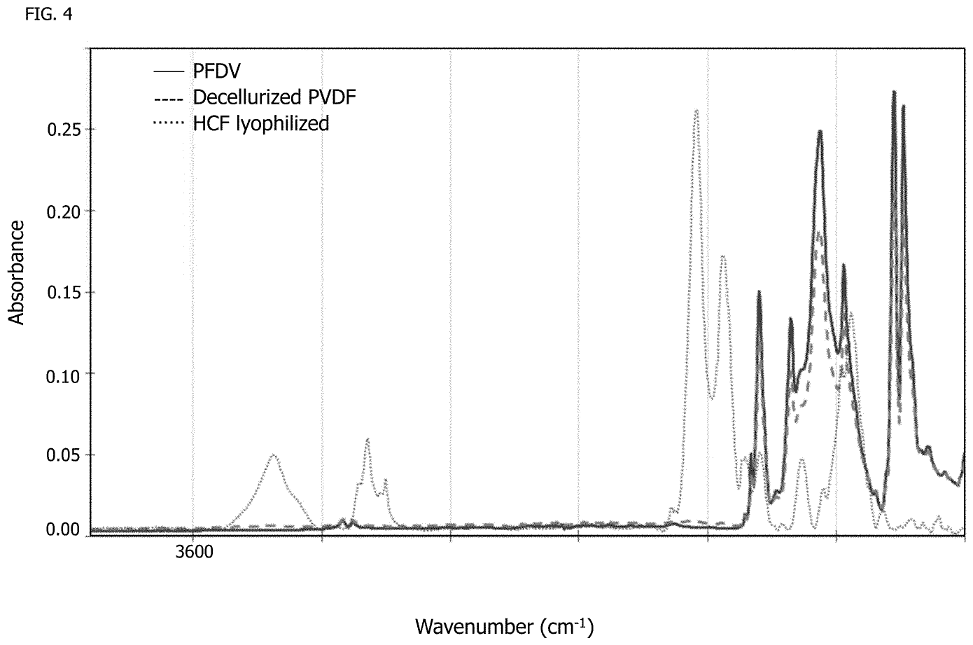

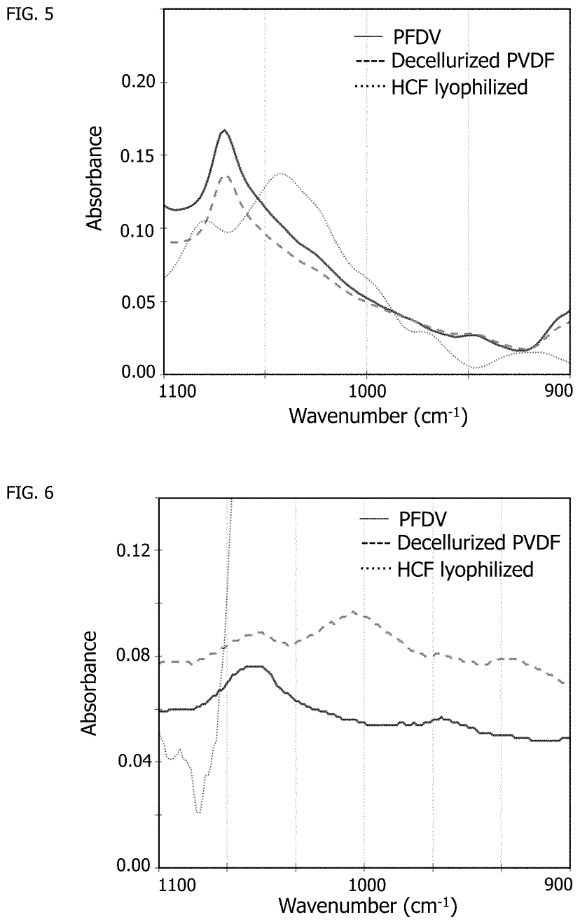

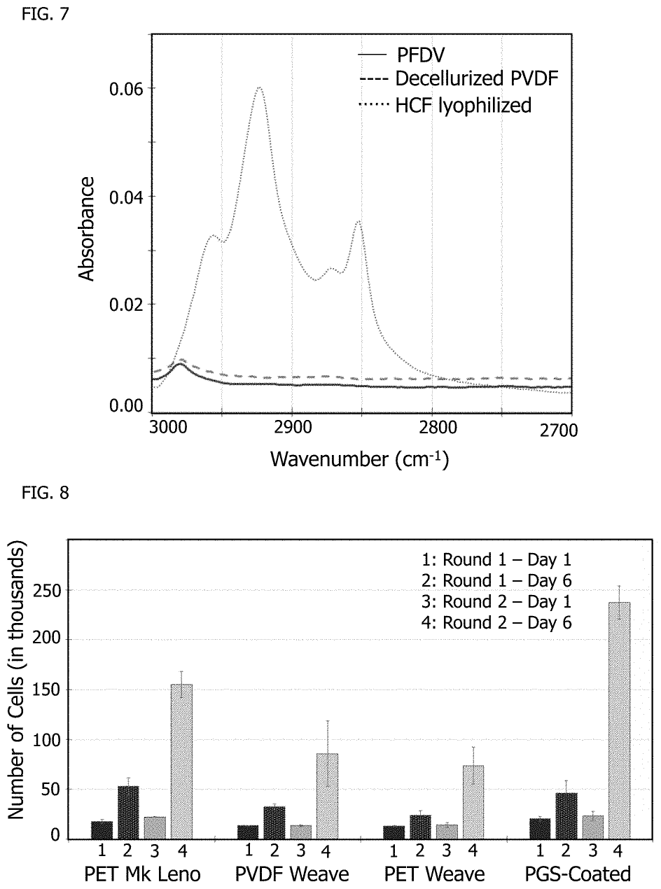

Fourier transform infrared (FTIR) spectroscopy was used to characterize both the uncoated PVDF woven textile and decellularized, coated PVDF woven textile of Example 1 along with lyophilized human cardiac fibroblast (HCF) cells to chemically identify the deposition of extracellular matrix by cells. FIG. 4 shows the full spectra of the three samples. FIG. 5 shows a magnified region of interest of wavenumber 900 cm⁻¹ to 1100 cm⁻¹, where a C—O stretch bond indicative of the presence of polysaccharides at wavenumber 1041 cm⁻¹ is seen with lyophilized cells but is not present with native PVDF textile or decellularized PVDF, indicating the cells are no longer present in the decellularized textile of Example 1. FIG. 6 shows another magnified region of interest of wavenumber 1500 cm⁻¹ to 1800 cm⁻¹, where two amide peaks at wavenumbers 1653 cm⁻¹ and 1544 cm⁻¹ for the decellularized PVDF of Example 1 are indicative of the presence of proteinaceous deposited ECM by cardiac fibroblasts cultured on the woven PVDF textile. FIG. 7 shows yet another magnified region of interest of wavenumber 2700 cm⁻¹ to 3000 cm⁻¹, where CH₂ and CH₃ stretch peaks of lipids contained in the cell membrane are present at wavenumbers 2757 cm⁻¹, 2929 cm⁻¹, and 2853 cm⁻¹ when characterizing lyophilized cells but are not present in the native PVDF textile and decellularized PVDF of Example 1.

### Example 3

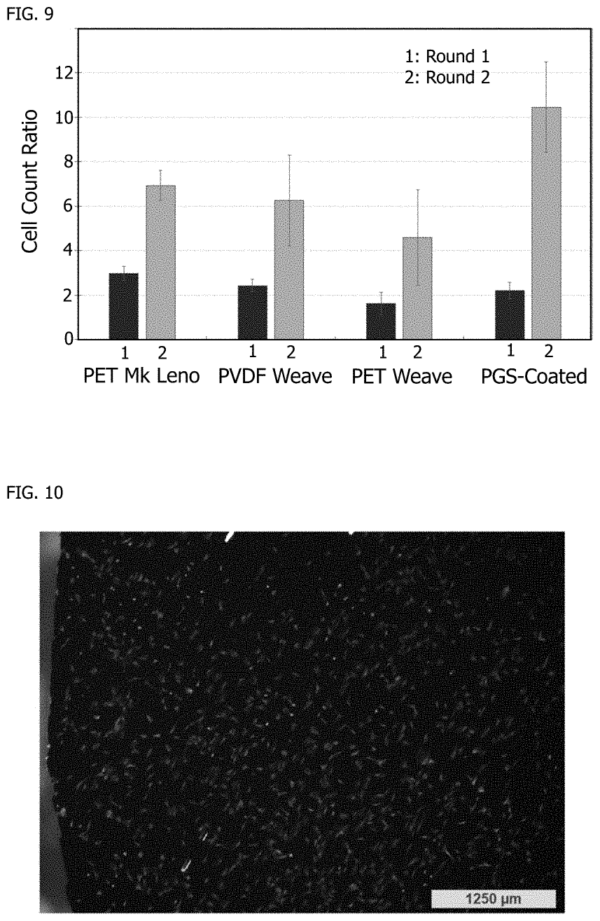

Similar cell cultures to those described in Example 1 were done on a PET mock leno weave, on a PET plain weave, and on a PGA textile coated with PGS. During the cell cultures on the four different samples, an alamarBlue® assay was used to calorimetrically determine cell counts by associating the cell proliferation rate measured by the assay against a standard curve of known cell dilutions using a UV-vis spectrometer to determine cell count after one and six days of culture over two rounds separate of cultures, the results of which are shown in FIG. 8. The assays demonstrated that increased levels of proliferation occur during the second-round cardiac fibroblast culture, when the cells are cultured on the decellularized textile scaffolds coated with ECM deposited by cardiac fibroblasts during the first round of cell culture. FIG. 9 shows that the proliferation factor, a ratio of the cell count at day 6 to the cell count at day 1, is notably higher in the second round, when cardiac fibroblasts are cultured on the human cell-deposited ECM coated textile scaffolds in comparison to the native scaffolds of the first round regardless of the specific textile being used.

### Example 4

The PGS-coated PGA textile disk of Example 3, ECM-coated as a result of cell culture with cardiac fibroblasts, was stained with Calcein AM and imaged by a fluoresce microscope. Calcein AM is a live cell tracker that fluoresces in the cell cytosol of viable cells. FIG. 10 shows the resulting fluorescence microscopy image. The light coloration indicates live cardiac fibroblasts and the dark coloration indicates areas of the ECM-coated textile disk that are free of live cells. The image shows normal spreading and attachment of cardiac fibroblasts on the ECM-coated, PGS-coated PGA textile disk.

All references cited herein are hereby incorporated by reference in their entirety.

While the foregoing specification illustrates and describes exemplary embodiments, it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A process of forming a coated textile comprising:
culturing human cells on at least one fiber of a textile such that the human cells produce and deposit human extracellular matrix (hECM) on the textile; and
removing the human cells from the hECM to provide the coated textile as the textile and a coating comprising a residual of the hECM produced and deposited by the human cells on the textile during the culturing.

2. The process of claim 1, wherein the human cells are human fibroblast cells.

3. The process of claim 1, wherein the human cells are autograft cells extracted from a human in need of the coated textile.

4. The process of claim 1 further comprising modifying a surface of the at least one fiber to promote cell adhesion to the surface prior to the culturing.

5. The process of claim 1 further comprising modifying a surface of the at least one fiber to increase hECM production by the human cells.

6. The process of claim 5, wherein the modifying comprises coating the surface of the at least one fiber with a solid-state bioreactor composition comprising a poly(glycerol sebacate) adduct, wherein the poly(glycerol sebacate) adduct comprises poly(glycerol sebacate) and a promoting factor or a promoting factor precursor.

7. The process of claim 1 further comprising coating the at least one fiber of the textile with a layer of poly(glycerol sebacate) prior to culturing the human cells on the at least one fiber.

8. The process of claim 1 further comprising culturing a second type of human cells on the textile after the removing.

9. The process of claim 1 further comprising extracting the human cells from a human and purifying the human cells prior to the culturing.

10. A coated textile comprising:
a textile; and
a coating on the textile, the coating comprising human extracellular matrix (hECM), wherein the hECM is in a cell-deposited state in the coating.

11. The coating of claim 10, wherein the textile comprises a polymeric material selected from the group consisting of poly(ethylene terephthalate), polytetrafluoroethylene, polypropylene, polyethylene, polyethylene vinyl acetate, collagen, poly(glycolic acid), poly(glycerol sebacate), poly(lactic acid), poly(lactic-co-glycolic acid), poly(trimethylene carbonate), and polycaprolactone.

12. The coated textile of claim 10, wherein the textile is woven, braided, non-woven, or knit.

13. The coated textile of claim 10, wherein the hECM is selected from the group consisting of a human proteoglycan, human heparan sulfate, human chondroitin sulfate, human keratan sulfate, a human non-proteoglycan polysaccharide, human hyaluronic acid;
human collagen, human elastin, human fibronectin, human laminin, and combinations thereof.

14. The coated textile of claim 10, wherein the coated textile is free of xenogenic material.

15. The coated textile of claim 10, wherein the coated textile is decellularized.

16. The coated textile of claim 10, wherein the coating further comprises a layer of poly(glycerol sebacate) on the textile.

17. The coated textile of claim 10, wherein the textile comprises at least one fiber comprising a glycerol-sebacate-containing polymer.

18. The coated textile of claim 10, wherein the hECM is from autograft cells extracted from a human in need of the coated textile.

19. A solid-state bioreactor composition comprising a poly(glycerol sebacate) adduct, wherein the poly(glycerol sebacate) adduct comprises poly(glycerol sebacate) and a promoting factor or a promoting factor precursor.

20. A method comprising:
implanting a coated textile in a human, wherein the coated textile is an autograft comprising a textile and a coating on the textile, the coating comprising human extracellular matrix (hECM) in a cell-deposited state deposited by human cells from the human.
